(19) United States
(12) Patent Application Publication
(10) Pub. No.: US 2003/0166263 A1
(43) Pub. Date: Sep. 4, 2003

(54) Microfabricated spotting apparatus for producing low cost microarrays

(76) Inventors: Robert C. Haushalter, Los Gatos, CA (US); Xiao-Dong Sun, Fremont, CA (US)

Correspondence Address: DUANE MORRIS LLP, 100 COLLEGE ROAD WEST, SUITE 100, PRINCETON, NJ, 08540-6604, US

(21) Appl. No.: 10/220,913

(22) PCT Filed: Feb. 6, 2002
(86) PCT No.: PCT/US02/03974

(51) Int. Cl. C12M 1/34; G01D 15/04; G01D 15/20

(52) U.S. Cl. 435/287.2; 346/78

(57) ABSTRACT

An improved apparatus for producing microarrays of chemical and biochemical materials. The apparatus includes one or more pins, a holder for the pins and a dispensing tray for holding one or more liquids. The apparatus is microfabricated from semiconductor materials such as silicon, silicone oxides, silicon carbide, silicon nitride, polymers, ceramics, non-ferric alloys using chemical and physical microfabrication and photolithographic techniques.

This application claims the benefit of U.S. Provisional Application No. 60/266,609 filed Feb. 6, 2001.

### FIELD OF THE INVENTION

This invention relates to microarrays, and more particularly, to an apparatus for producing microarrays comprising high precision pins, a pin holder, and optionally, a multi-well dispensing tray, and methods for microfabricating the apparatus.

### BACKGROUND OF THE INVENTION

Microarray technology is emerging as one of the principal and fundamental investigational tools for a very wide variety of biological problems. Although the preparation of DNA microarrays for use in many types of analysis is one of the main applications today, it is clear that the basic idea of easily obtaining huge amounts of data from a rapid and relatively simple to use platform is set to penetrate most areas of biology and may find comparably broad use in chemistry and materials science. Such diverse areas of biology as genetics, population biology, immunology, rational drug design, genetic engineering and therapies, protein engineering, developmental biology and structural biology would all benefit from a rapid infusion of an inexpensive version of microarray technology. As with many other areas of technology, the true power of the technique will only become fully utilized when it is efficiently coupled to other related or complementary technology. For example, the coupling of the speed, ease and cost of microarray technology to amplification techniques could allow an approximately “real time” look into the biochemical machinery and mechanisms of a single cell as a function of time after various biochemical challenges.

In order to derive maximum benefit from a young technology area such as that of microarrays, the technology needs to be simple, inexpensive to purchase and use and be of reasonable physical size. For microarray technology, this translates into a system that should give better performance than the best current system, in a more compact format at a much lower price.

As shown in FIG. 1, there are six basic and common steps for most microarray-based experiments. After defining the question or problem to be addressed by the microarray based experiment, a sample (which is often a DNA oligomer) is bound to a substrate, which is normally a glass slide treated with a reagent capable of covalently bonding the DNA to the glass substrate. The sample is then applied to the substrate.

There are three common methods used for application of the sample to the substrate, each with its own compliment of advantages and disadvantages. The important parameters for various dispensing devices are summarized in Table I below.

TABLE I

| | | | Solenoid/ |
|---|---|---|---|
| Parameter | Microspotting Pin | Piezoelectric/Inkjet | Syringe |
| Spots/mm² | 4-100 | 4-25 | 2-4 |
| Volume | 0.5-2.5 | 0.05-10 | 5-200 |
| printed (nL) | | | |
| Adjustable | Need separate pin | Yes | Yes |
| volume | | | |
| Spot size | 75-400 | 120-180 | 250-500 |
| (μm) | | | |
| Spots/second | 64 | ˜500 | ˜40 |
| Robustness | Higher | Lower | Intermediate |
| Cost/spot | Least | Most | Intermediate |
| Loading volume | 0.2-1.0 | 10 | 10 |
| of dispensing | | | |
| device (μL) | | | |

It is clear from the data in Table 1 that microspotting pins are a competitive technology in terms of speed, quality and cost. Accordingly, a large portion of these arrays are accomplished with high precision metal microspotting pins. Unfortunately, the metal microspotting pins are individually machined at costs up to $400 each. The high cost of the pins prohibit many laboratories from using microspotting pin technology. Moreover, the metal pins are susceptible to bending damage and complex features which may further the utility of the pins can not be fabricated using traditional machine shop fabrication methods.

Therefore, in order to increase the usefulness of microspotting pin technology and make it even more attractive, low cost microspotting pins with improved performance are needed.

### BRIEF DESCRIPTION OF THE DRAWINGS

In the drawings, the like reference characters have been used to identify like elements.

FIG. 1 is a flow chart depicting the six basic and common steps for most microarray-based experiments.

FIG. 2 is an elevational view of a section of a microfabricated, spotting apparatus according to an exemplary embodiment of the present invention;

FIG. 3A is an elevational view of a spotting pin according to an exemplary embodiment of the present invention;

FIG. 3B is a side elevational view of the spotting pin of FIG. 3B;

FIG. 3C is a view of the dispensing tip section of the spotting pin of FIG. 3A;

FIG. 4A is an enlarged view the dispensing tip section according to a first exemplary embodiment of the present invention;

FIG. 4B is an enlarged view the dispensing tip section according to a second exemplary embodiment of the present invention;

FIG. 4C is an enlarged view the dispensing tip section according to a third exemplary embodiment of the present invention;

FIG. 5A is a plan view of a section of a holder according to an exemplary embodiment of the present invention;

FIG. 5B is an elevational view of the holder;

FIG. 6A is an elevational view of the holder according to a second exemplary embodiment of the present invention;

FIG. 6B are elevational views depicting the operational advantage of the holder of FIG. 6A;

FIG. 7 is an elevational view of a section of a dispensing tray according to an exemplary embodiment of the present invention;

FIGS. 8A-8G depict the microfabrication of the pins and holders according to an exemplary embodiment of the present invention; and

FIGS. 9A-9F depict the microfabrication of the dispensing tray according to an exemplary embodiment of the present invention.

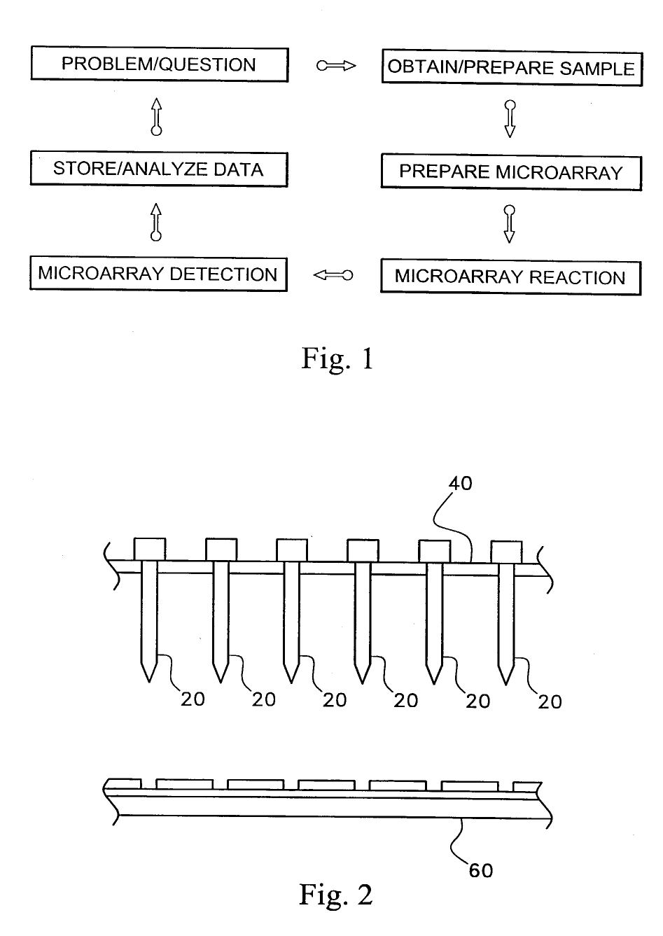

It should be understood that the drawings are solely for the purpose of illustrating the concepts of the invention and are not intended as a level of the limits of the invention.

### DETAILED DESCRIPTION

FIG. 2 shows a microfabricated, spotting apparatus according to an exemplary embodiment of the present invention, comprising the following microfabricated components: one or more high precision microspotting pins 20, a pin holder 40, and optionally, a multi-well dispensing tray 60. The apparatus of the present invention especially useful for printing and manufacturing high quality microarrays of proteins, DNA, RNA, polypeptides, oligonucleotides and microarrays of other biological materials. The microspotting apparatus of the present invention may also be used for printing and manufacturing high quality microarrays of other matters, such as solid semiconductor quantum dots or liquid dots containing various functional molecules, such as sensors.

The components 20, 40, 60 of the apparatus may be composed of any material or combination of materials suitable for microfabrication including but not limited to semiconductor materials such as silicon (Si), silicon carbide (SiC), silicon nitride (Si₃N₄), insulator materials such as silicon dioxide (SiO₂), polymers, ceramics, non-ferric alloys. Any suitable microfabrication method or combination of methods may be used for making the components 20, 40, 60, depending upon the material or materials selected for the components 20, 40, 60, the desired dimensional precision of the components 20, 40, 60, and/or the desired manufacturing yield. Suitable microfabrication methods include but are not limited to chemical and physical microfabrication, photolithography, photoresist methods, micro-electromechanical methods, e-beam lithography, and x-ray lithography. Precision machining techniques including but not limited to EDM and laser cutting may be used to supplement the microfabrication methods.

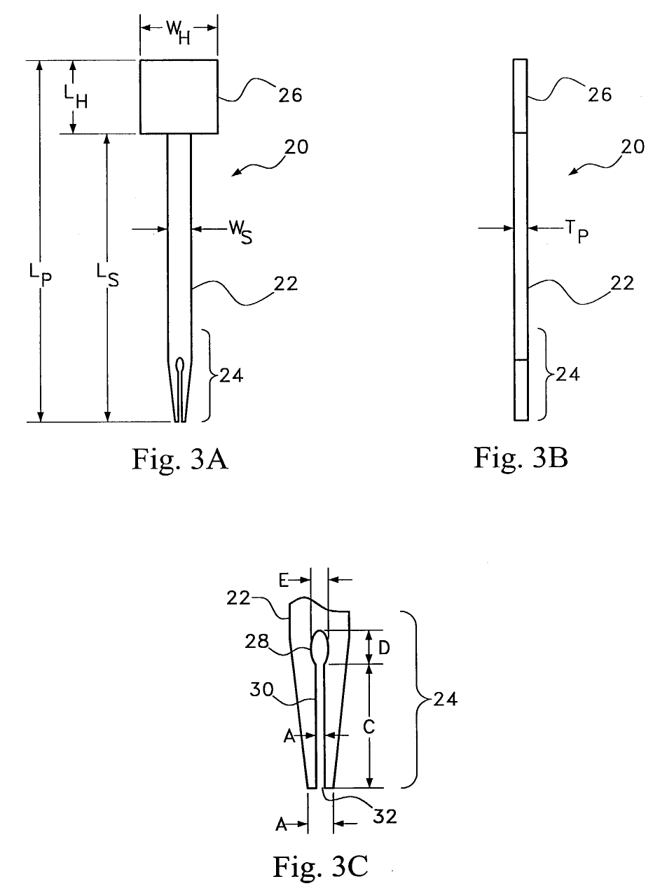

FIGS. 3A-3C collectively illustrate an exemplary embodiment of the high precision, spotting pin 20 of the present invention. The pin 20 typically includes a generally rectangular shaft 22 with a printing tip section 24, and an enlarged, generally rectangular pin mounting head 26 disposed at the end of the shaft 22 opposite the printing tip section 24. The pin 20 may have a length Lₚ anywhere between about 10 μm and 100 mm and a thickness Tₚ anywhere between about 10 μm and 10 mm. The mounting head 26 may have a length L_{H} anywhere between about 2 μm and 20 mm and a width W_{H} anywhere between about 2 μm and 10 mm. The shaft 22 may have a length L_{S} anywhere between about 8 μm and 80 mm and a width W_{S} anywhere between about 2 μm and 10 mm. In one illustrative example, the pin 20 may have a length L_{P} of about 6 mm and a thickness T_{P} of about 200 μm, the mounting head 26 may have a length L_{H} of about 1 mm and a width W_{H} of about 1 mm, and the shaft 22 may have a length L_{S} of about 5 mm and a width W_{S} of about 500 μm.

One of ordinary skill in the art will of course appreciate that the shape and dimensions of the pin 20 may be varied. For example, the rectangular shaft 22 prevents the pin 20 from rotating in correspondingly shape micromachined slots 42 in the pin holder 40 as will be explained later. In other embodiments of the invention, the shaft 22 can be square, or be cylindrical and provided with other means which prevents rotation in the pin holder 40.

As best shown in FIG. 3C, formed essentially in the printing tip section 24 of the shaft 22 is a generally elliptical shaped aperture or sample holding reservoir 28, and an elongated slot or channel 30 that communicates with the sample holding reservoir 28 and extends to a dispensing tip 32 of the shaft 22. The slot 30 enables a liquefied sample to be drawn into and stored in the sample holding reservoir 28 and then be dispensed at the dispensing tip 32 of the shaft 22.

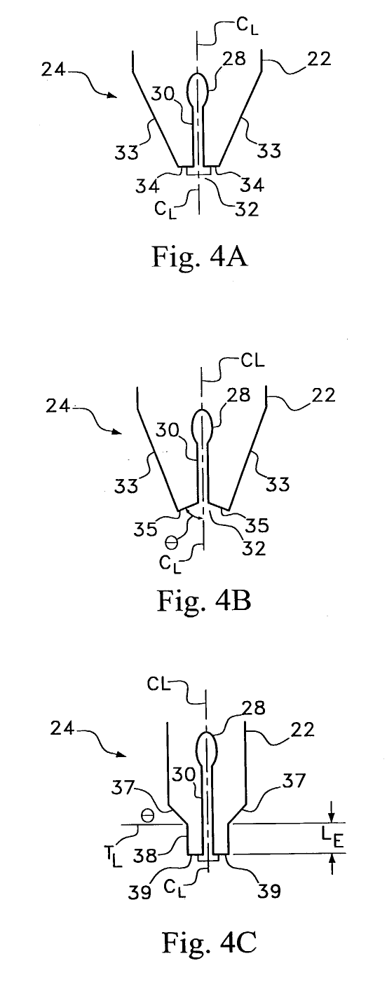

The structures of the printing tip section 24 including but not limited to the reservoir 28, channel 30, and/or the dispensing tip 32 are configured and dimensioned to optimized the microspotting process. FIG. 4A shows a first exemplary embodiment of the printing tip section 24 of the shaft 22. The printing tip section 24 of this embodiment is formed with two side wall surfaces 33 that gradually taper toward the dispensing tip 32. The dispensing tip 32 is formed by two substantially flat printing end wall surfaces 34 oriented generally perpendicular to the center line CL of the pin 20, such that the surfaces 34 are generally parallel to the surface of a substrate to be printed.

FIG. 4B shows a second exemplary embodiment of the printing tip section 24 of the shaft 22. The printing tip 24 of this embodiment is also formed with two side wall surfaces 33 that gradually taper toward the dispensing tip 32. However, the dispensing tip 32 is formed by two substantially flat, inwardly facing printing end wall surfaces 35, that are each oriented at an acute angle θ relative to the center line CL of the pin 20, thereby defining a cavity 36 therebetween. The cavity 36 provides a slightly larger tip volume than the embodiment of the tip shown in FIG. 4A. The larger tip volume may provide a slightly larger touch off volume per head print area, which may improve the shape and volume of the resultant spot. The larger tip volume may allow the same amount of liquid to be deposited with a lighter than normal touch-off pressure.

FIG. 4C shows a third exemplary embodiment of the printing tip section 24 of the shaft 22. The printing tip section 24 of this embodiment is formed with two side wall surfaces 37 that quickly taper down (each at an angle θ of each about 60 ° measured relative to a transverse line TL which is perpendicular to the center line CL of the pin20 ), and then extend parallel to one another thereby defining a non-tapered portion 38 that extends to the dispensing tip 32. The non-tapered portion may have a length LE of about 500 μm in one exemplary embodiment. The dispensing tip 32 is defined by two substantially flat printing end wall surfaces 39 oriented generally perpendicular to the center line CL of the pin20. The printing tip section 24 of this embodiment is intended to reduce clinging of the spotting solution to the side wall surfaces 37 thereby producing a smaller, cleaner and more well defined spot.

As mentioned earlier, the configuration and dimensions of the printing tip section 24 can be easily varied, via the above described microfabrication methods, to optimize the microspotting process. As shown in FIG. 3C, the area A of the dispensing tip (formed by the two printing end wall surfaces) that touches the substrate may be between about 10^{-6 μm} ² and 10 mm², the width B of the channel may be between about 1 μm and 300 μm, the length C of the channel may be between about 2 μm and 2 mm, and the major axis D of the reservoir may be between about 1.5 μm and 8 mm and the minor axis E of the reservoir may be between about 2 μm and 600 μm. In one illustrative embodiment, the area A of the dispensing tip may be about 4 mm². The width B of the channel may be between about 75 μm and 100 μm and the length C of the channel may be between about 400 μm and 2000 μm. The major axis D of the reservoir may be between about 200 μm and 1000 μm and the minor axis E of the reservoir may be between about 40 μm and 200 μm.

The configuration and dimensions of the printing tip section 24 can be adjusted so that the volume of liquid sample deposited by each pin 20 and/or the area of the spotted liquid sample (spot) can be varied as desired. It is contemplated that For example the configuration and dimensions of the printing tip section 24 can be adjusted so that the volume of liquid sample deposited by each pin 20 can be as large as about 0.1 milliliters (mL), as minute as about 10-4 picoliter (pL), or any volume between about 0.1 mL and 10⁻⁴ pL. Similarly, the configuration and dimensions of the printing tip section 24 can be adjusted so that the area of the spotted liquid sample (spot) deposited by each pin 20 can be as large as about10 square millimeters (mm²), as minute as about 10⁻⁶ square microns (μm²), or any area between about 10 mm² and about 10⁻⁶ μm². There are trade-offs among these dimensions that must be balanced. For instance, increasing the dimensions of the major and minor axes of the reservoir 28 to increase the volume thereof in order to decrease the number of fill steps can compromise the mechanical stability of the pin shaft 22.

One of ordinary skill in the art will of course appreciate that the printing tip section 24 may be configured in various other ways to optimize the microspotting process. For example, the surface or surfaces making up the dispensing tip 32 may be smooth, textured, concave, convex, include one or more pores, channels, or nozzles or combinations of the same. Further, the printing tip section 24 may be designed such that the entire shaft 22 of the pin 20 does not have to be submersed into the stock solution to be spotted, thereby obviating the time and material wasting pre-spotting procedure.

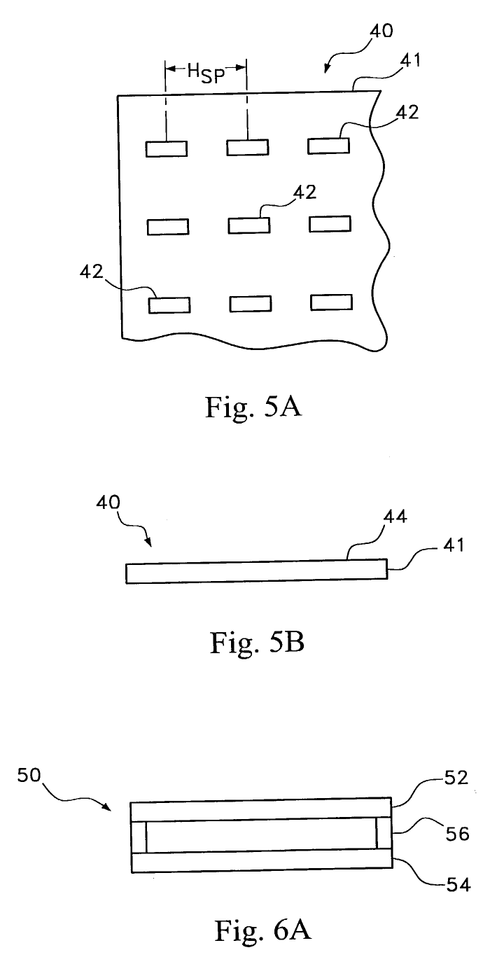

FIGS. 5A and 5B illustrate an exemplary embodiment of the pin holder 40 of the present invention. The pin holder 40 is typically configured as a planar member 41 having an array of rectangular, microfabricated slots 42 extending therethrough, each of the slots 42 accepting a pin 20 of the present invention. The configuration and dimensions of the pin holder 40 may be varied accommodate up to 100,000 pins 20 of the present invention. In one illustrative embodiment, the holder may be 10 cm by 16 cm. The configuration and dimensions of the slots 42 may also be adjusted to provide a pin density, i.e., the number of pins per unit area of the holder, of about 1 pin per 10 mm² of holder area to about 10⁶ pins per mm² of holder area. The pin density of the holder 40 is important as it determines the spot density of the resulting microarray produced by the holder 40 and pin 20 assembly. The slots 42 of the pin holder40 are also configured and dimensioned to allow the shafts 22 of the pins 20 to be slip-fitted into the slots 42 in a frictionless manner with no lateral movement, and suspended by their mounting heads 26, which rest on the upper surface 44 of the pin holder 40, while preventing rotation of the pins 20 in the slots 42.

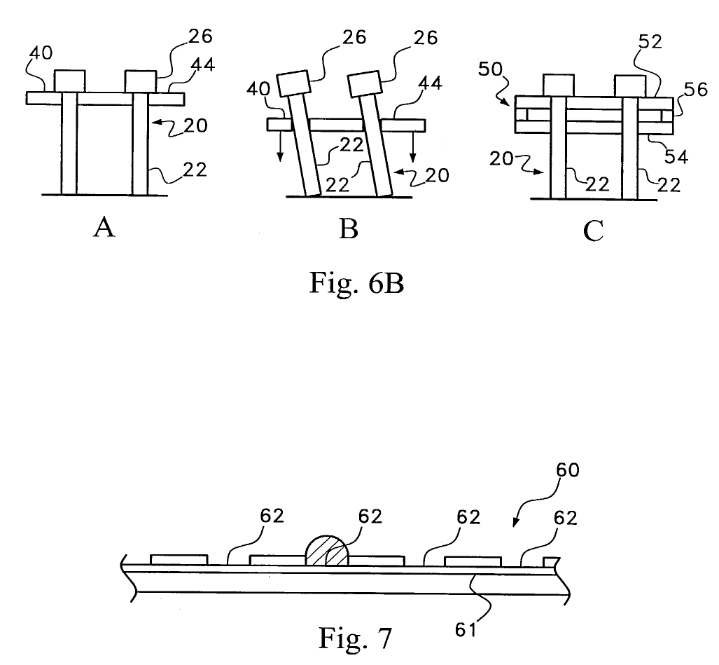

FIG. 6A illustrates a second exemplary embodiment of the pin holder 50 of the present invention. In this embodiment, upper and lower pin holders 52, 54 are bonded together by a perimeter spacer 56 in a single unit referred to herein as a collimating holder. The collimating holder 50 is used to prevent the pins 20 from “tipping over” when touching the substrate as shown in FIG. 6B. More specifically, when the pins 20 touch the substrate surface during printing, the pins 20 may be excessively raised out of the “non-collimated” holder 40 of the previous embodiment such that the mounting heads 26 no longer touch the upper surface 44 of the holder member 41 to prevent the pins 20 from tipping over. The collimating holder 50 solves this problem by providing the lower holder 54, which guides the bottom portion of the pin shafts 22 to maintain the vertical orientation of the pins 20 in the holder 50.

In the exemplary embodiment of FIG. 5A, 1536 slots 42 may be provided in the holder 40 (or in the upper and lower pin holders 52, 54 of the holder 50 of FIG. 6A) and the slots 42 may have a center-to-center spacing H_{SP} of 2.25 mm. One of ordinary skill in the art will recognize that this embodiment of the pin holder may be advantageously used with a conventional 1536 well microtiter plate (which holds the sample solutions and is not shown herein), as the wells of the microtiter plate have the same 2.25 mm center-to-center spacing as the slots of this exemplary pin holder. Hence, 1536 pins can be installed in the pin holder and dipped directly into all 1536 wells of the microtiter plate, or, with every other pin removed, into a conventional 384 well microtiter plate (which has a 4.5 mm center-to-center well spacing).

Table II below shows a comparison of the cycle time to make copies of high density microarrays for print heads with increasing number of spotting pins. As can be seen there is a dramatic reduction of cycle time to print copies of microarrays with an increased number of pins in the print head. Each printing cycle includes loading pins, preprinting, printing arrays, and washing, with typical estimated times for each step. Basically the time for each step is the same for increasing number of pins, and the time to make microarray copies is inversely proportional to the number of pins in the head. A printhead using the present invention's holder and pin assembly with, for example, 96 microfabricated Si pins, dramatically reduces the time to make copies (48) of high density (60×384=23,040 spots) microarray.

TABLE II

| | Time | | | |
|---|---|---|---|---|
| Steps | 1 pin | 8 pins | 32 pins | 96 pins (Si-pin) |
| Load pins | 2 sec | 2 sec | 2 sec | 2 sec |
| with sample | | | | |
| Preprint 10x | 4 sec | 4 sec | 4 sec | 4 sec |
| Print 48 slides | 48 sec | 48 sec | 48 sec | 48 sec |
| Wash pins | 6 sec | 6 sec | 6 sec | 6 sec |
| Total for 1 Cycle | 1 min | 1 min | 1 min | 1 min |
| Total for 1 × 384 | 384 min | 48 min | 12 min | 4 min |
| well plate | | | | |
| Total for 60 × 384 | 384 hour | 48 hour | 12 hour | 4 hour |
| well plate | | | | |

Additional increases in microarray printing speed can be realized using the multi-well dispensing tray 60 of the present invention. FIG. 7 illustrates an exemplary embodiment of the multi-well dispensing tray of the present invention. The dispensing tray 60 is typically configured as a planar member 61 having an array of microfabricated sample holding wells 62 defined in the member61. The configuration and dimensions of the dispensing tray 60 may be varied accommodate up to 100,000 wells 62. The configuration and dimensions of the wells 62 may also be varied to provide a well density, i.e., the number of well per unit area of the dispensing tray, of about 1 well per 10 mm of dispensing tray area to about 10⁶ wells per mm² of dispensing tray area.

As described earlier, the components 20, 40, 60 of the apparatus may be composed of any material or combination of materials suitable for microfabrication including but not limited to semiconductor materials such as Si, SiC, SiO₂, Si₃N₄, polymers, ceramics, non-ferric alloys, although Si is preferred. Also, any suitable microfabrication method or combination of methods may be used for making the components 20, 40, 60, depending upon the material or materials selected for the components 20, 40, 60, the desired dimensional precision of the components 20, 40, 60, and/or the desired manufacturing yield.

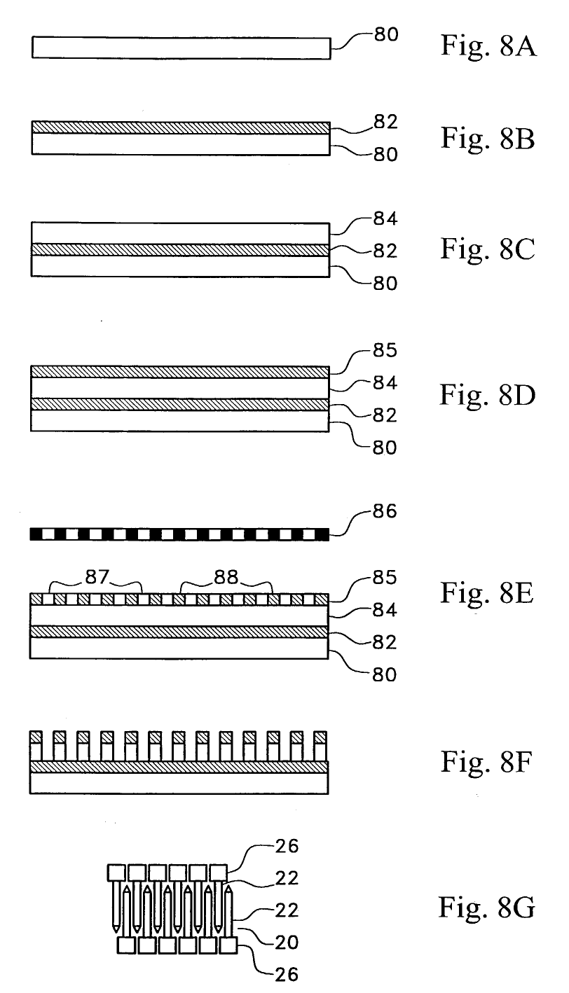

FIGS. 8A-8G illustrate the microfabrication of silicon-based pins and holders according to an exemplary embodiment of the present invention using conventional silicon microfabrication methods. First, pin and holder design data is used to design a photomask (FIG. 8E). The design of the photomask may be prepared using any suitable CAD software program, such as AutoCAD®. The photomask may then be prepared, for example, by generating a negative image of the design in chromium on a long wavelength UV transparent glass substrate.

As shown in FIGS. 8A and8B, a first layer of photoresist 82 may be deposited onto a first silicon wafer 80. The first silicon wafer 80 may be made from single crystal silicon having a (100) crystal orientation, with both sides polished and about 200 μm thick. The first layer of photoresist 82 may be deposited, for example, using a conventional spin coating technique.

In FIG. 8C, a second silicon wafer 84 (component wafer 84 ) is bonded on top of the first silicon wafer 80 (support wafer 80 ) by placing the second wafer 84 on top of the first layer of photoresist 82 and soft-baking the first layer of photoresist 82 for about 1 and 2 minutes at approximately 90 °. The second silicon wafer 80 may also be made from single crystal silicon having a (100) crystal orientation, with both sides polished and about 200 μm thick. The first layer of photoresist 82 between the wafers 80, 84 prevents severe undercutting of the component wafer 84 when etchant travels therethrough. Such an etchant is used when, for example, Reactive Ion Etching (RIE) micromachining is used. One of ordinary skill in the microfabrication art will of course recognize that any other suitable bonding material or method may be used to bond the two wafers 80, 84 together.

As shown in FIG. 8D, a second layer of photoresist 85 is deposited over the component wafer 84 and soft-baked. The second layer of photoresist 85 layer is patterned as shown in FIG. 8E, by placing the photomask over the second layer of photoresist 85, irradiating the wafers 80, 84 and developing the second layer of photoresist 85. The irradiated portions 87 of the second layer of the photoresist 85 are removed from the component wafer 84, thus, leaving a photoresist pattern thereon, which is made up of the non-irradiated regions of photoresist 88.

In FIG. 8F, the pins and holders are micromachined from the component wafer 84 using any conventional silicon micromachining technique, such as RIE. As is well known in the silicon microfabrication art, the micromachining process removes the portions of the silicon wafer not protected by the photoresist.

The general layout of the pins 20 on a section of the component wafer 84 is shown in FIG. 8G. As can be seen mounting heads 26 of the pins 20 may be packed closely together with the shafts 22 filling most of the space when the pins 20 are formed in an interdigitated pattern. This efficient space filling allows the maximum number of pins to be fabricated per unit area of wafer surface.

The component wafer 84 is machined all the way through as shown in FIG. 8F to separate the pins and holders. The separated pin and holders are removed from the support wafer 80 by dissolving the first and layer of photoresist 82 with solvent (the solvent also removes the patterned sections 88 of the second layer of photoresist 85 from the components). After several thorough washings in fresh solvent, the separated pin and holder components are oxidized using conventional well known silicon oxidizing methods to form a thin (typically about 1 μm thick) SiO₂ hydrophilic film layer on the components. At this stage, the pins and holder may be assembled.

The smoothness (rms roughness) of the RIE cut surfaces are typically well below 1μ, and 5 μfeatures are easy to fabricate. Most of the exposed surface of the pin, which corresponds to the polished surfaces of the wafer covered by photoresist during the RIE treatment, has a roughness only in the tens of Angstroms. This smoothness abrogates the need for the shaft-polishing step required for the steel, which is necessary for the shaft to slide freely in its holder. Since the holder for the silicon pins is also microfabricated, the high tolerances and smooth surfaces allow for a high precision, but smooth fit during the movement of the pin in the holder during printing. Accordingly, the pins and holders have a very smooth, mirror like finish and slide without restriction. Although the machining accuracy of each pin is important, it is also imperative that the uniformity of all pins manufactured is accordingly as high. Batch-to-batch uniformity is one of the great strengths of silicon microfabrication and typically all the components are essentially identical yielding more uniform microarrays. The fabrication of complex pin shapes and the cutting of intricate features into the pins are simple with this fabrication technique, limited only by the achievable feature size, limitations of the cutting technique and the mechanical strength of the part.

The pins and holders may be assembled together by placing a desired number of the pins into each of the holders. This may be accomplished with the aid of a vacuum tweezers, which grasps the mounting head of the pin. Each pin is dropped into a desired slot in the holder with the aid of a small plastic funnel that guides the pin into the slot.

After the pins are placed in a corresponding holder, the holder is attached to the arm of a precision x-y-z motion control system (not shown). The pins are moved to the source plate location and the pins filled by dipping onto the solution. The volume picked up by each pin is on the order of a microliter or less of solution. Since this small volume can rapidly evaporate, thereby producing a concomitant change in the concentration in the solution to be deposited, the entire apparatus must be contained within a humidity-controlled chamber. The spot is actually made by the careful z motion and touching the pins to the substrate. To account for height variations on the surface of the substrate, and to prevent unnecessary wear on the pin tips, the pins “float” in their holder and rise out of the slots of the holder as they touch the surface of the substrate as shown in FIG. 6B, thereby providing a very light touch, but one that depends on the weight of the pin. After the pins are filled, they go through a “pre-spotting” procedure of ˜20 spottings during which time the volume deposited decreases to its steady state value. Presumably, this is due to the removal of the liquid film that is adhering to the external surface of the shaft with subsequent print volumes replenished by drawing from the reservoir28 and channel30. The pin transfers the print volume to a hydrophobic or chemically reactive surface to prevent spreading of the drop. In many cases the material being deposited is also (reversibly) covalently bound to the surface of the substrate, for example the nitrogen functionality on the DNA oligomer bound to an aldehyde group of the substrate to form a Schiff base complex that is later removable. After deposition the subsequent processing depends on the final application but in the case of DNA oligomers, the surface is used in hybridization experiments with a probe DNA molecule.

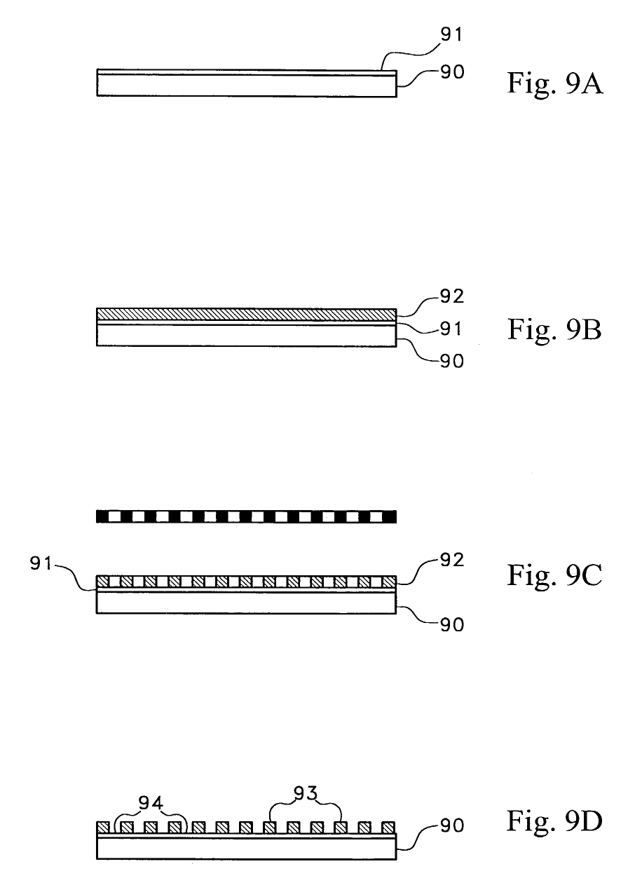

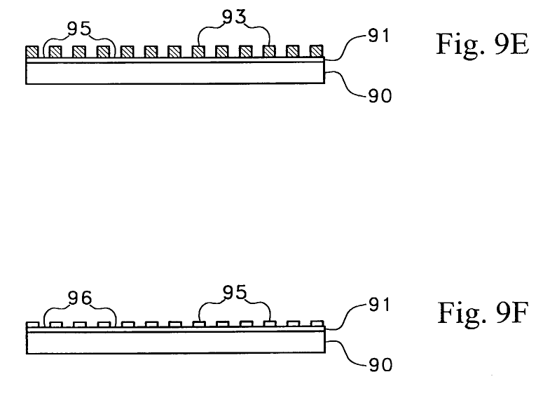

FIGS. 9A- 9F illustrate the microfabrication of one or more silicon-based, 384 well dispensing trays according to an exemplary embodiment of the present invention using conventional silicon microfabrication methods. Starting with a silicon wafer 90, a thin (typically about 1 μxm thick) silicon dioxide layer 91 is formed on the wafer 90 using conventional methods as shown in FIG. 9A.

In FIGS. 9B-9D, a layer of photoresist 92 is deposited over the SiO₂ layer 91 and patterned to generate areas of photoresist 93 that are 2 mm in diameter on 4.5 mm centers, with exposed areas 94 of SiO₂ between them.

Next, the exposed portions 94 of the SiO₂ layer of the wafer surface are silanized to render them hydrophobic as shown in FIG. 9E, and then, the photoresist areas 93 are dissolved. This leaves 2 mm diameter regions 96 of exposed SiO₂ , which are hydrophilic, surrounded by the silanized portions 95 as shown in FIG. 9F. Finally, the wafer is separated into plurality of individual dispensing trays. Drops of various solutions (samples), each with a volume of approximately 4 μL, can be easily deposited by a liquid handling robot onto each of the hydrophilic areas 96 and will bead up and become perfectly localized in hydrophilic region as shown in FIG. 7.

Table III below shows the performance characteristics of the spotting apparatus of the present invention should far exceed those of current state-of-the-art spotting systems. Advantages of microfabricated spotting pins, especially those made from silicon, over machined metal pins include 10-100 fold higher dimensional tolerances, less than 1% of the weight (lighter pressure gives more uniform spots), tip hardness, the ability to chemically modify the Si0₂ surface of the pins to control wetting and liquid uptake/release, higher pin density in array (higher spot density in microarray), more precise volumetric uptake into pin, lower surface friction (ease of sliding movement in holder), resistance of tip to bending damage and the ability to fabricate complex features not obtainable by traditional machine shop fabrication. The combination of increased tip hardness and lower pin weight should translate into far less wear on the tip. The decreased tip wear will result in a more uniform spot deposited as the tip ages. The thin shafts on the Si pins, combined with the far greater elasticity of Si versus steel, suggest that the Si pins will not suffer from bending damage. The deposited drop size from the Si pin should be more uniform than those from the steel pins not only because of the higher tolerances, greater uniformity of machined dimensions from pin-to-pin and slower tip wear but the precision of the volumetric uptake of liquid into the pin should be higher as well.

Multiple wafers can be processed simultaneously to further cut the cost of production of the pins, holders and dispensing trays. It is estimated that a 100 to 1000 fold reduction in price per pin, hence placing direct contact printing methodologies within the economic reach of most laboratories and greatly expand the number and quality of experiments that can be performed.

TABLE III

| Property | Silicon | Stainless Steel |
|---|---|---|
| Machining | In microns | In mils |
| tolerances/feature size | | |
| Smoothness of surface | Very smooth as fabricated | Requires |
| | | polishing |
| Ease of mass production | Facile parallel fabrication | Made one |
| | | at a time |
| Pin-to-pin uniformity | Extremely high | Lower and |
| As manufactured | | relatively much |
| | | more variable |
| Relative weight of pins | Proposed Si pin weigh 1% | 100 fold higher |
| | of steel pin; lighter touch to | weight per |
| | substrate = better spots | pin |
| Ease of complex feature | All features made at once | Each feature |
| fabrication | | individually |
| | | machined |
| Surface friction sliding | Lower | Higher |
| against other materials | | |
| Hardness of tip material | Harder | Softer |
| Resistance of pin tip to | Higher | Lower |
| bending damage | | |
| Deposited drop size and | Since tip has smaller, more | Larger, more |
| uniformity | precise features, smaller | irregular spots |
| | more uniform spots result | |
| Precision of volumetric | Higher | Lower |
| uptake into tip | | |
| Packing density of pins | Since pins can be made | Lower packing |
| into holder, ie microarray | smaller, there are more pins | density of |
| spot density | per unit area | larger pins |
| Chemical resistance | Very good | Good |
| Methods known to | Extensive chemistry | Less well |
| chemically modify surface | of SiO₂ | developed |
| | surfaces known | |
| Cost per pin | <$1 (estimated) | ˜$400 |

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An apparatus for producing a microarray, the apparatus comprising:
a pin for depositing a predetermined volume of a liquid on a substrate, the pin including:
a dispensing tip at a first end thereof; and
a reservoir communicating with the dispensing tip;
wherein the pin is microfabricated from a material selected from the group consisting of semiconductors, polymers, ceramics, and non-ferric alloys.

2. The apparatus according to claim 1, further comprising:
a holder for touching the pin to the substrate to deposit the predetermined volume of the liquid on the substrate, the holder including:
a first planar member having a first aperture extending therethrough for receiving the pin;
wherein the holder is microfabricated from a material selected from the group consisting of semiconductors, polymers, ceramics, and non-ferric alloys.

3. The apparatus according to claim 2, wherein the holder further includes:
a second planar member having a second aperture extending therethrough for receiving a bottom portion of the pin, the second planar member disposed under the first planar member such that the apertures are in axial alignment with one another.

4. The apparatus according to claim 2, wherein the aperture in the first planar member of the holder and the pin include a surface arrangement that prevents rotation of the pin in the aperture.

5. The apparatus according to claim 2, wherein the pin further includes:
a head disposed at a second end thereof that engages the first planar member of the holder to prevent the pin from falling through the holder.

6. The apparatus according to claim 2, wherein the pin comprises a plurality of pins and the aperture in the first planar member of the holder comprises an array of apertures, each of the apertures for receiving one of the pins.

7. The apparatus according to claim 6, further comprising:
a dispensing tray having an array of wells which each hold a liquid;
wherein the tray is microfabricated from a material selected from the group consisting of semiconductors, polymers, ceramics, and non-ferric alloys.

8. The apparatus of according to claim 7, wherein the holder defines a predetermined aperture density, the dispensing tray defining a predetermined well density that is equal to the predetermined aperture density of the holder.

9. The apparatus according to claim 2, wherein the pin comprises a plurality of pins and the aperture in the first planar member of the holder comprises an array of up to 32 apertures, each of the apertures for receiving one of the pins.

10. The apparatus according to claim 2, wherein the pin comprises a plurality of pins and the aperture in the first planar member of the holder comprises an array of up to 64 apertures, each of the apertures for receiving one of the pins.

11. The apparatus according to claim 2, wherein the pin comprises a plurality of pins and the aperture in the first planar member of the holder comprises an array of up to 128 apertures, each of the apertures for receiving one of the pins.

12. The apparatus according to claim 2, wherein the pin comprises a plurality of pins and the aperture in the first planar member of the holder comprises an array of up to 500 apertures, each of the apertures for receiving one of the pins.

13. The apparatus according to claim 2, wherein the pin comprises a plurality of pins and the aperture in the first planar member of the holder comprises an array of up to 1,000 apertures, each of the apertures for receiving one of the pins.

14. The apparatus according to claim 2, wherein the pin comprises a plurality of pins and the aperture in the first planar member of the holder comprises an array of up to 5,000 apertures, each of the apertures for receiving one of the pins.

15. The apparatus according to claim 2, wherein the pin comprises a plurality of pins and the aperture in the first planar member of the holder comprises an array of up to 10,000 apertures, each of the apertures for receiving one of the pins.

16. The apparatus according to claim 2, wherein the pin comprises a plurality of pins and the aperture in the first planar member of the holder comprises an array of up to 100,000 apertures, each of the apertures for receiving one of the pins.

17. The apparatus according to claim 2, wherein the pin comprises a plurality of pins and the aperture in the first planar member of the holder comprises an array of apertures, each of the apertures for receiving one of the pins, the array of apertures having an aperture density of about 1 aperture 10 mm , the aperture density providing a maximum pin density of about1 pin per 10 mm², the pin density determining a density of the resulting microarray.

18. The apparatus according to claim 2, wherein the pin comprises a plurality of pins and the aperture in the first planar member of the holder comprises an array of apertures, each of the apertures for receiving one of the pins, the array of apertures having an aperture density between about 0.1 and 1 aperture/mm², the aperture density providing a maximum pin density between about 0.1 and 1 pin/mm², the pin density determining a density of the resulting microarray.

19. The apparatus according to claim 2, wherein the pin comprises a plurality of pins and the aperture in the first planar member of the holder comprises an array of apertures, each of the apertures for receiving one of the pins, the array of apertures having an aperture density between about 1 and 10 aperture/mm , the aperture density providing a maximum pin density between about 1 and 10 pin/mm , the pin density determining a density of the resulting microarray.

20. The apparatus according to claim 2, wherein the pin comprises a plurality of pins and the aperture in the first planar member of the holder comprises an array of apertures, each of the apertures for receiving one of the pins, the array of apertures having an aperture density between about 10 and 100 aperture/mm , the aperture density providing a maximum pin density between about 10 and 100 pin/mm², the pin density determining a density of the resulting microarray.

21. The apparatus according to claim 2, wherein the pin comprises a plurality of pins and the aperture in the first planar member of the holder comprises an array of apertures, each of the apertures for receiving one of the pins, the array of apertures having an aperture density between about 100 and 1000 aperture/mm², the aperture density providing a maximum pin density between about 100 and 1000 pin/mm² , the pin density determining a density of the resulting microarray.

22. The apparatus according to claim 2, wherein the pin comprises a plurality of pins and the aperture in the first planar member of the holder comprises an array of apertures, each of the apertures for receiving one of the pins, the array of apertures having an aperture density between about 10³ and 10⁴ aperture/mm², the aperture density providing a maximum pin density between about 10³ and 10⁴ pin/mm², the pin density determining a density of the resulting microarray.

23. The apparatus according to claim 2, wherein the pin comprises a plurality of pins and the aperture in the first planar member of the holder comprises an array of apertures, each of the apertures for receiving one of the pins, the array of apertures having an aperture density between about 10⁴ and 10⁵ aperture/mm², the aperture density providing a maximum pin density between about 10⁴ and 10⁵ pin/mm², the pin density determining a density of the resulting microarray.

24. The apparatus according to claim 2, wherein the pin comprises a plurality of pins and the aperture in the first planar member of the holder comprises an array of apertures, each of the apertures for receiving one of the pins, the array of apertures having an aperture density between about 10⁵ and 10⁶ aperture/mm², the aperture density providing a maximum pin density between about 10⁵ and 10^{6 pin/mm}², the pin density determining a density of the resulting microarray.

25. The apparatus according to claim 2, further comprising:
a dispensing tray having a well for holding a liquid;
wherein the tray is microfabricated from a material selected from the group consisting of semiconductors polymers, ceramics, and non-ferric alloys.

26. The apparatus according to claim 1, further comprising:
a dispensing tray having a well for holding a liquid;
wherein the tray is microfabricated from a material selected from the group consisting of semiconductors, polymers, ceramics, and non-ferric alloys.

27. The apparatus according to claim 26, wherein the well of the dispensing tray comprises an array of wells for holding liquids, the array of wells having a well density of about 1 well/ 10 mm².

28. The apparatus according to claim 26, wherein the well of the dispensing tray comprises an array of wells for holding liquids, the array of wells having a well density between about 0.1 and 1 well/mm².

29. The apparatus according to claim 26, wherein the well of the dispensing tray comprises an array of wells for holding liquids, the array of wells having a well density between about 1 and 10 well/mm².

30. The apparatus according to claim 26, wherein the well of the dispensing tray comprises an array of wells for holding liquids, the array of wells having a well density between about 10 and 10² well/mm².

31. The apparatus according to claim 26, wherein the well of the dispensing tray comprises an array of wells for holding liquids, the array of wells having a well density between about 10² and 10³ well/mm².

32. The apparatus according to claim 26, wherein the well of the dispensing tray comprises an array of wells for holding liquids, the array of wells having a well density between about 10³ and 10⁴ well/mm².

33. The apparatus according to claim 26, wherein the well of the dispensing tray comprises an array of wells for holding liquids, the array of wells having a well density between about 10⁴ and 10⁵ well/mm².

34. The apparatus according to claim 26, wherein the well of the dispensing tray comprises an array of wells for holding liquids, the array of wells having a well density between about 10⁵ and 10⁶ well/mm .

35. The apparatus according to claim 1, wherein the pin further includes:
a channel for transferring the liquid between the reservoir and the dispensing tip.

36. The apparatus according to claim 1, wherein the predetermined volume comprises about 0.1 mL.

37. The apparatus according to claim 1, wherein the predetermined volume comprises between about 0.1 mL and 10 μL.

38. The apparatus according to claim 1, wherein the predetermined volume comprises between about 1 μL and 10 μL.

39. The apparatus according to claim 1, wherein the predetermined volume comprises between about 100 nL and 1000 nL.

40. The apparatus according to claim 1, wherein the predetermined volume comprises between about 10 nL and 100 nL.

41. The apparatus according to claim 1, wherein the predetermined volume comprises between about 1 nL and 10 nL.

42. The apparatus according to claim 1, wherein the predetermined volume comprises between about 100 pL and 1000 pL.

43. The apparatus according to claim 1, wherein the predetermined volume comprises between about 10 pL and 100 pL.

44. The apparatus according to claim 1, wherein the predetermined volume comprises between about 1 pL and 10 pL.

45. The apparatus according to claim 1, wherein the predetermined volume comprises between about 0.1 pL and 1 pL.

46. The apparatus according to claim 1, wherein the predetermined volume comprises between about 0.01 pL and 0.1 pL.

47. The apparatus according to claim 1, wherein the predetermined volume comprises about 10⁻² pL.

48. The apparatus according to claim 1, wherein the predetermined volume comprises between about 10⁻³ and 10⁻⁴ pL.

49. The apparatus according to claim 1, wherein the predetermined volume of the liquid deposited on the substrate forms a spot having an area of about 1 to 10 mm².

50. The apparatus according to claim 1, wherein the predetermined volume of the liquid deposited on the substrate forms a spot having an area of about 10⁵ μm².

51. The apparatus according to claim 1, wherein the predetermined volume of the liquid deposited on the substrate forms a spot having an area of between about 10⁴ and 10⁵μm².

52. The apparatus according to claim 1, wherein the predetermined volume of the liquid deposited on the substrate forms a spot having an area of between about 10³ and 10⁴μm².

53. The apparatus according to claim 1, wherein the predetermined volume of the liquid deposited on the substrate forms a spot having an area of between about 10² and 10³μm².

54. The apparatus according to claim 1, wherein the predetermined volume of the liquid deposited on the substrate forms a spot having an area of between about 10 and 10² μm².

55. The apparatus according to claim 1, wherein the predetermined volume of the liquid deposited on the substrate forms a spot having an area of between about 1 and 10 μm².

56. The apparatus according to claim 1, wherein the predetermined volume of the liquid deposited on the substrate forms a spot having an area of between about 10⁻¹ and 1 μm².

57. The apparatus according to claim 1, wherein the predetermined volume of the liquid deposited on the substrate forms a spot having an area of between about 10⁻¹ and 10⁻² μm².

58. The apparatus according to claim 1, wherein the predetermined volume of the liquid deposited on the substrate forms a spot having an area of between about 10⁻¹ and 10⁻² μm².

59. The apparatus according to claim 1, wherein the predetermined volume of the liquid deposited on the substrate forms a spot having an area of between about 10⁻³ and 10^{-4 μm}².

60. The apparatus according to claim 1, wherein the predetermined volume of the liquid deposited on the substrate forms a spot having an area of between about 10⁻⁴ and 10^{-5 μm}².

61. The apparatus according to claim 1, wherein the predetermined volume of the liquid deposited on the substrate forms a spot having an area of between about 10⁻⁵ and 10^{-6 μm}².

62. A pin for depositing a predetermined volume of a liquid on a substrate to produce a microarray, the pin comprising:
a dispensing tip at a first end thereof; and
a reservoir communicating with the dispensing tip,
wherein the pin is microfabricated from a material selected from the group consisting of semiconductors, polymers, ceramics, and non-ferric alloys.

63. The pin according to claim 62, further comprising:
a head disposed at a second end thereof that enables the pin to be handled.

64. The pin according to claim 62, fuither comprising:
a channel for transferring the liquid between the reservoir and the dispensing tip.

65. The pin according to claim 62, wherein the predetermined volume comprises about 0.1 mL.

66. The pin according to claim 62, wherein the predetermined volume comprises between about 0.1 mL and 10 μL.

67. The pin according to claim 62, wherein the predetermined volume comprises between about 1 μL and 10 μL.

68. The pin according to claim 62, wherein the predetermined volume comprises between about 100 nL and 1000 nL.

69. The pin according to claim 62, wherein the predetermined volume comprises between about 10 nL and 100 nL.

70. The pin according to claim 62, wherein the predetermined volume comprises between about 1 nL and 10 nL.

71. The pin according to claim 62, wherein the predetermined volume comprises between about 100 pL and 1000 pL.

72. The pin according to claim 62, wherein the predetermined volume comprises between about 10 pL and 100 pL.

73. The pin according to claim 62, wherein the predetermined volume comprises between about 1 pL and 10 pL.

74. The pin according to claim 62, wherein the predetermined volume comprises between about 0.1 pL and 1 pL.

75. The pin according to claim 62, wherein the predetermined volume comprises between about 0.01 pL and 0.1 pL.

76. The pin according to claim 62, wherein the predetermined volume comprises about 10⁻² pL.

77. The pin according to claim 62, wherein the predetermined volume comprises between about 10−3 and 10⁻⁴ pL.

78. The pin according to claim 62, wherein the predetermined volume of the liquid deposited on the substrate forms a spot having an area of about 1 to 10 mm².

79. The pin according to claim 62, wherein the predetermined volume of the liquid deposited on the substrate forms a spot having an area of about 10⁵ μm².

80. The pin according to claim 62, wherein the predetermined volume of the liquid deposited on the substrate forms a spot having an area of between about 10⁴ and 10⁵ μm².

81. The pin according to claim 62, wherein the predetermined volume of the liquid deposited on the substrate forms a spot having an area of between about 10³ and 10⁴ μm².

82. The pin according to claim 62, wherein the predetermined volume of the liquid deposited on the substrate forms a spot having an area of between about 10² and 10³ μm².

83. The pin according to claim 62, wherein the predetermined volume of the liquid deposited on the substrate forms a spot having an area of between about 10 and 10²μm².

84. The pin according to claim 62, wherein the predetermined volume of the liquid deposited on the substrate forms a spot having an area of between about 1 and 10 μm².

85. The pin according to claim 62, wherein the predetermined volume of the liquid deposited on the substrate forms a spot having an area of between about 10−1 and 1 μm².

86. The pin according to claim 62, wherein the predetermined volume of the liquid deposited on the substrate forms a spot having an area of between about 10−1 and 10−2μm².

87. The pin according to claim 62, wherein the predetermined volume of the liquid deposited on the substrate forms a spot having an area of between about 10−1 and 10−2μm².

88. The pin according to claim 62, wherein the predetermined volume of the liquid deposited on the substrate forms a spot having an area of between about 10−3 and 10−4μm².

89. The pin according to claim 62, wherein the predetermined volume of the liquid deposited on the substrate forms a spot having an area of between about 10−4 and 10−5μm².

90. The pin according to claim 62, wherein the predetermined volume of the liquid deposited on the substrate forms a spot having an area of between about 10−5 and 10−6μm².

91. A holder for use in producing a microarray, the holder comprising:
a first planar member; and
a first aperture extending through the planar member for receiving a pin that deposits a predetermined volume of a liquid on a substrate to produce the microarray;
wherein the holder is microfabricated from a material selected from the group consisting of semiconductors, polymers, ceramics, and non-ferric alloys.

92. The holder according to claim 91, further comprising:
a second planar member having a second aperture extending therethrough for receiving a bottom portion of the pin, the second planar member disposed under the first planar member such that the apertures are in axial alignment with one another.

93. The holder according to claim 91, wherein the aperture in the first planar member comprises an array of up to 32 apertures.

94. The holder according to claim 91, wherein the aperture in the first planar member comprises an array of up to 64 apertures.

95. The holder according to claim 91, wherein the aperture in the first planar member comprises an array of up to 128 apertures.

96. The holder according to claim 91, wherein the aperture in the first planar member comprises an array of up to 500 apertures.

97. The holder according to claim 91, wherein the aperture in the first planar member comprises an array of up to 1,000 apertures.

98. The holder according to claim 91, wherein the aperture in the first planar member comprises an array of up to 5,000 apertures.

99. The holder according to claim 91, wherein the aperture in the first planar member comprises an array of up to 10,000 apertures.

100. The holder according to claim 91, wherein the aperture in the first planar member comprises an array of up to 100,000 apertures.

101. The holder according to claim 91, wherein the aperture in the first planar member comprises an array of apertures having an aperture density of about1 aperture/10 mm², the aperture density providing a maximum pin density of about 1 pin per 10 mm², the pin density determining a density of the resulting microarray.

102. The holder according to claim 91, wherein the aperture in the first planar member comprises an array of apertures having an aperture density between about 0.1 and 1 aperture/mm², the aperture density providing a maximum pin density between about 0.1 and 1 pin/mm², the pin density determining a density of the resulting microarray.

103. The holder according to claim 91, wherein the aperture in the first planar member comprises an array of apertures having an aperture density between about 1 and 10 aperture/mm^{2 , the aperture density providing a maximum pin density between about} 1 and 10 pin/mm², the pin density determining a density of the resulting microarray.

104. The holder according to claim 91, wherein the aperture in the first planar member comprises an array of apertures having an aperture density between about 10 and 100 aperture/mm², the aperture density providing a maximum pin density between about 10 and 100 pin/mm², the pin density determining a density of the resulting microarray.

105. The holder according to claim 91, wherein the aperture in the first planar member comprises an array of apertures having an aperture density between about 100 and 1000 aperture/mm², the aperture density providing a maximum pin density between about 100 and 1000 pin/mm², the pin density determining a density of the resulting microarray.

106. The holder according to claim 91, wherein the aperture in the first planar member comprises an array of apertures having an aperture density between about 10³ and 10⁴ aperture/mm², the aperture density providing a maximum pin density between about 10³ and 10⁴ pin/mm², the pin density determining a density of the resulting microarray.

107. The holder according to claim 91, wherein the aperture in the first planar member comprises an array of apertures having an aperture density between about 10⁴ and 10⁵ aperture/mm², the aperture density providing a maximum pin density between about 10⁴ and pin/mm², the pin density determining a density of the resulting microarray.

108. The holder according to claim 91, wherein the aperture in the first planar member comprises an array of apertures having an aperture density between about 10⁵ and 10⁶aperture/mm², the aperture density providing a maximum pin density between about 10⁵ and 10⁶ pin/mm², the pin density determining a density of the resulting microarray.

109. A dispensing tray for use in producing a microarray, the dispensing tray comprising:
a well for holding a liquid;
wherein the tray is microfabricated from a material selected from the group consisting of semiconductors, polymers, ceramics, and non-ferric alloys.

110. The dispensing tray according to claim 109, wherein the well of the dispensing tray comprises an array of wells for holding liquids, the array of wells having a well density of about 1 well/10 mm².

111. The dispensing tray according to claim 109, wherein the well of the dispensing tray comprises an array of wells for holding liquids, the array of wells having a well density between about 0.1 and 1 well/mm².

112. The dispensing tray according to claim 109, wherein the well of the dispensing tray comprises an array of wells for holding liquids, the array of wells having a well density between about 1 and 10 well/mm².

113. The dispensing tray according to claim 109, wherein the well of the dispensing tray comprises an array of wells for holding liquids, the array of wells having a well density between about 10 and 10² well/mm².

114. The dispensing tray according to claim 109, wherein the well of the dispensing tray comprises an array of wells for holding liquids, the array of wells having a well density between about 10² and 10³ well/mm².

115. The dispensing tray according to claim 109, wherein the well of the dispensing tray comprises an array of wells for holding liquids, the array of wells having a well density between about 10³ and 10⁴ well/mm².

116. The dispensing tray according to claim 109, wherein the well of the dispensing tray comprises an array of wells for holding liquids, the array of wells having a well density between about 104 and 105 well/mm².

117. A method of making a pin for depositing a predetermined volume of a liquid on a substrate to produce a microarray, the pin having a dispensing tip at a first end thereof and a reservoir communicating with the dispensing tip, the method comprising:
selecting at least one material from the group consisting of semiconductors, polymers, ceramics, and non-ferric alloys;
generating design parameters of the pin; and
fabricating the pin from the selected at least one material in accordance with the generated design parameters using at least one technique selected from the group consisting of photolithography, photoresist technology, micro electromechanical system technology, laser cutting, water jet cutting, electronic discharge machine cutting, and precision micromaching.

118. A method of making a holder for use in producing a microarray, the holder having a first planar member and a first aperture extending through the planar member for receiving a pin that deposits a predetermined volume of a liquid on a substrate to produce the microarray, the method comprising:
selecting at least one material from the group consisting of semiconductors, polymers, ceramics, and non-ferric alloys;
generating design parameters of the holder; and
fabricating the holder from the selected at least one material in accordance with the generated design parameters using at least one technique selected from the group consisting of photolithography, photoresist technology, micro electromechanical system technology, laser cutting, water jet cutting, electronic discharge machine cutting, and precision micromaching.

119. A method of making a dispensing tray for use in producing a microarray, the dispensing tray having a well for holding a liquid, the method comprising:
selecting at least one material from the group consisting of semiconductors, polymers, ceramics, and non-ferric alloys;
generating design parameters of the dispensing tray; and
fabricating the dispensing tray from the selected at least one material in accordance with the generated design parameters using at least one technique selected from the group consisting of photolithography, photoresist technology, micro electromechanical system technology, laser cutting, water jet cutting, electronic discharge machine cutting, and precision micromaching.

120. A microarray made with the apparatus according to claim 1, the microarray comprising a biological reagent selected from the group consisting of proteins, polypeptides, amino acids, DNA, oligonucleotides, RNA, and mixtures of molecules from 70 to 76.

121. A microarray made with the pin according to claim 62, the microarray comprising a biological reagent selected from the group consisting of proteins, polypeptides, amino acids, DNA, oligonucleotides, RNA, and mixtures of molecules from 70 to 76.

122. A microarray made with the apparatus according to claim 1, the microarray comprising a functional solid state material.

123. The microarray according to claims 122, wherein the functional solid state material comprises quantum dots.

124. A microarray made with the pin according to claim 62, the microarray comprising a functional solid state material.

125. The microarray according to claims 124, wherein the functional solid state material comprises quantum dots.

126. A microarray made with the apparatus according to claim 1, the microarray comprising a functional liquid dots.

127. The microarray according to claims 126, wherein the functional liquid dots comprise sensors.

128. A microarray made with the pin according to claim 62, the microarray comprising a functional liquid dots.

129. The microarray according to claims 128, wherein the functional liquid dots comprise
